(19) United States
(12) Patent Application Publication
(10) Pub. No.: US 2021/0130202 A1
(43) Pub. Date: May 6, 2021

(54) WATER TREATMENT SYSTEM USING OZONE

(71) Applicants: CLEAN TEAM HOLDINGS INC., Pasadena, CA (US); ECLEAN TECHNOLOGIES, LLC, La Canada, CA (US)

(72) Inventors: Alejandro Islas, San Gabriel, CA (US); William Lance Burrows, Big Bear Lake, CA (US)

(21) Appl. No.: 16/970,540

(22) PCT Filed: Jan. 25, 2019
(86) PCT No.: PCT/US2019/015288

§ 371 Date: Aug. 17, 2020

Related U.S. Application Data
(60) Provisional application No. 62/631,769, filed on Feb. 17, 2018.

(51) Int. Cl. C02F 1/78; C02F 1/00; B01F 3/04; A61L 2/26; A61L 2/20

(52) U.S. Cl. CPC C02F 1/78; A61L 2101/02; B01F 3/04241; B01F 3/04439; A61L 2/26; A61L 2/202; C02F 2201/782; C02F 2303/04; C02F 2201/784; C02F 2201/005; C02F 2209/40; B01F 2003/04319; B01F 2003/0434; B01F 2003/04347; B01F 2003/04886; B01F 2215/0052; C02F 1/001

(57) ABSTRACT

A water treatment system using ozone is disclosed. The system includes a container for holding water, an ozone supply configured to supply ozone, and a water treatment device secured at a lower portion of the container. The water treatment device is configured to disinfect the water using ozone in the container. The water treatment device includes a housing having a plurality of openings and a tortuous porous tube in the housing. The tortuous porous tube is configured to receive the ozone from the ozone supply and distribute the ozone into the water.

### PRIORITY CLAIM

This application claims priority to and the benefit of U.S. provisional patent application No. 62/631,769 filed in the United States Patent and Trademark Office on Feb. 17, 2018, the entire content of which is incorporated herein by reference as if fully set forth below in its entirety and for all applicable purposes.

### FIELD

The disclosure relates, in some aspects, to a water treatment system. More specifically, but not exclusively, the disclosure relates to a water treatment system using ozone.

### BACKGROUND

Ozone contains three atoms of oxygen (O₃) that is different than the two atoms oxygen (O₂) in breathable air (hereafter “air”). Ozone may also be known as “activated oxygen” or O₃. The oxidizing properties of ozone can reduce or eliminate taste and odor problems in air or water. Furthermore, ozone is a powerful sterilant that can be used as an effective disinfectant to treat air, water, or other fluids. In some applications, ozone effectively kills bacteria, viruses, microorganisms, spores, and many other pathogens, while it removes dissolved organic materials by oxidative processes. Ozone has been used to treat and disinfect water supply.

### BRIEF SUMMARY OF SOME EXAMPLES

The following presents a simplified summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

One aspect of the present disclosure provides a water treatment system using ozone. The system includes a container for holding water, an ozone supply configured to supply ozone, and a water treatment device secured at a lower portion of the container. The water treatment device is configured to disinfect the water using ozone in the container. The water treatment device includes a housing having a plurality of openings and a tortuous porous tube in the housing. The tortuous porous tube is configured to receive the ozone from the ozone supply and distribute the ozone into the water.

In some aspects of the present disclosure, the tortuous porous tube may have a coiled shape, a helical shape, a spiral shape, a circular shape, or a wound shape. In some aspects of the disclosure, the tortuous porous tube when extended is substantially longer than the water treatment device in at least one of a length direction or width direction.

Another aspect of the present disclosure provides a method of treating water using ozone. The method fills water into a container through a water filter secured to an upper portion of the container. Then, the method disinfects the filtered water using a water treatment device located at a lower portion of the container. The water treatment device includes a housing having a plurality of openings and a tortuous porous tube in the housing, the tortuous porous tube is configured to distribute ozone into the filtered water.

### BRIEF DESCRIPTION OF THE DRAWINGS

FIGS. 1 and 2 are conceptual diagrams illustrating a water treatment system using ozone according to one aspect of the disclosure.

FIG. 3 is a diagram illustrating a water treatment system with two water treatment devices according to another aspect of the disclosure.

FIG. 4 is a diagram illustrating an ozone supply according to one aspect of the disclosure.

FIGS. 5 and 6 are diagrams illustrating a water treatment device according to one aspect of the disclosure.

FIG. 7 is a diagram illustrating a water treatment device with two porous tubes according to one aspect of the disclosure.

FIG. 8 is a diagram illustrating a water treatment device with a porous tube in a coiled shape according to one aspect of the disclosure.

### DETAILED DESCRIPTION

Referring now to the drawings, embodiments of water treatment systems and methods for treating water using ozone as a sterilant or disinfectant are disclosed in the present disclosure. The disclosed water treatment system has the working components being self-contained such that the system is transportable and easy to operate.

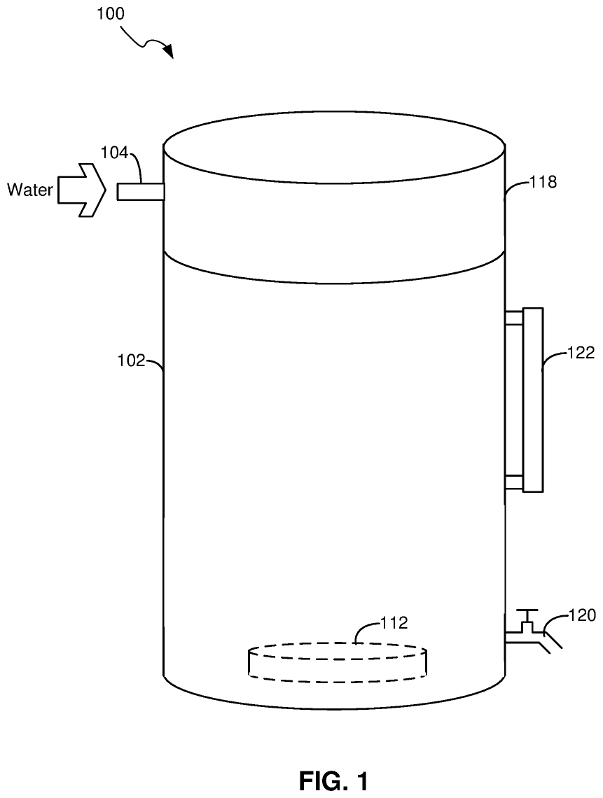

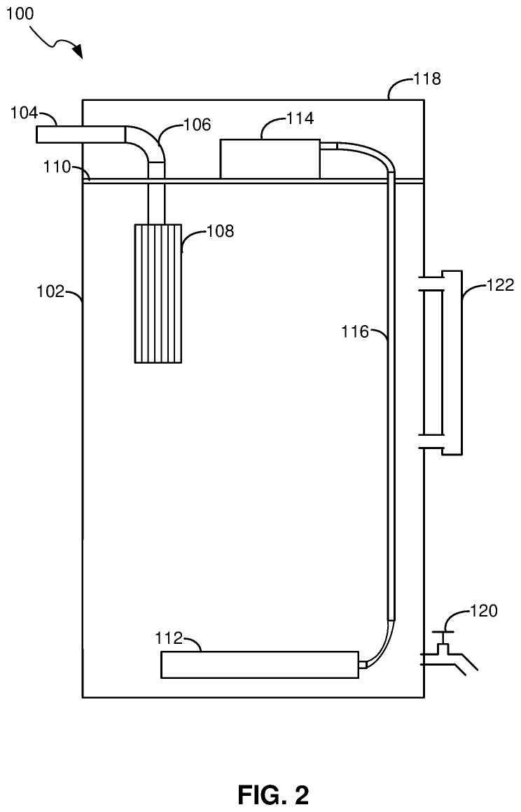

FIGS. 1 and 2 are conceptual diagrams illustrating a water treatment system 100 using ozone according to one aspect of the disclosure. FIG. 1 is a perspective view of the assembled water treatment system 100. FIG. 2 is a side view illustrating some components located inside the water treatment system 100.

The water treatment system 100 includes a container 102 for holding water or any desired fluid. The container 102 may be made of any suitable material, for example, plastic, metal, composite material, fiberglass, or wood. The container 102 has an inlet 104 for receiving water. In some examples, a rigid or flexible pipe 106 (see FIG. 2) may be connected between the inlet 104 and a water filter 108 in the container 102. Water entering the container 102 is filtered by the water filter 108 that may be located at a top or upper portion of the container 102. For example, the water filter 108 may be suspended from a lid or cover 110. In some aspects of the disclosure, the water filter 108 is configured to filter sediment, undesirable chemicals, biological contaminants, and/or suspended solids from the water. In one aspect of the disclosure, the water filter 108 may be a pleated sediment filter or the like. In some aspects of the disclosure, the water filter 108 may be removable such that it can be replaced as often as needed. In some aspects of the disclosure, the water filter 108 may be optional. In that case, water may enter the container 102 without filtering.

A water treatment device 112 is located at the bottom or lower portion of the container 102 when it is in an orientation or position to store water. The water treatment device 112 is configured to discharge, distribute, or diffuse an effective amount of ozone (O₃) into the water stored in the container 102. In some aspects of the disclosure, one or more such water treatment devices may be installed in the container. When multiple water treatment devices are used, they may be distributed in the container 102, for example, at different heights relative to the bottom of the container. The ozone distributed by the water treatment device 112 treats and disinfects the water, making it safer for human consumption. For example, the water treatment device 112 can discharge an effective amount air mixture containing a certain volume percentage of ozone. In one example, the air mixture may have an ozone concentration up to about 0.3 part per million (ppm).

An ozone supply 114 for providing ozone (e.g., O₃) is located on the upper portion of the container 102, for example, on a lid or cover 110. In operation, the water treatment device 112 receives a supply of ozone or ozone-mixed air from the ozone supply 114 via a pneumatically sealed hose, pipe, or tube 116 of any suitable length and/or shape. In some aspects of the disclosure, the water treatment system 100 may not include the ozone supply 114. Instead, an external source (not shown) may supply ozone or ozone-mixed air to the water treatment device 112. A removable cover 118 may be installed on top of the container 102 to provide protection for the ozone supply 14 and other components. The cover 118 may be made of any suitable material or shape. The cover 118 can be easily removed to allow access to the ozone supply 14 and other components of the system.

In some aspects of the disclosure, a valve 120 (e.g., spigot) may be fitted near the bottom or lower portion of the container 102 to control the release of the disinfected water. In some aspects of the disclosure, the water treatment system 100 may include a water level indicator 122 that is in fluid communication with the container 102. For example, at least a portion of the water level indicator 122 is transparent that can show the water level in the container 102.

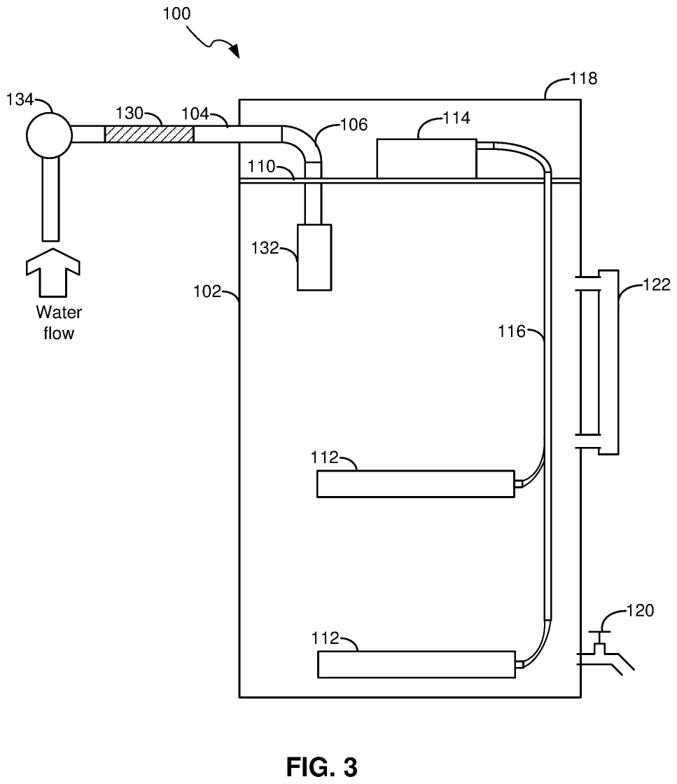

FIG. 3 is a diagram illustrating another configuration of the water treatment system 100 according to another aspect of the disclosure. In this example, the water treatment system has two or more water treatment devices 112 that may be placed at different positions (e.g., different heights) in the container 102. An inline water filter 130 may be located outside of the container 102 and connected to the inlet 104. Locating the water filter 130 outside the container allows easier service and replacement as needed. The water treatment system 100 may include a fill valve 132 that is configured to prevent water and/or ozone gas from escaping the container 102 as backflow. In some aspects of the disclosure, the fill valve 132 may be configured to automatically regulate and control the flow of water into the container 102. For example, when fill value 132 detects that the water level is below is predetermined level in the container 102, the fill value 132 automatically opens to allow water flowing into the container 102. Then, when the fill value 132 detects that the water level is at or above the predetermined level, the fill valve 312 automatically closes to stop the water flow. In some aspects of the disclosure, the water treatment system 100 may include a gauge 134 that is configured to measure the amount of water flow.

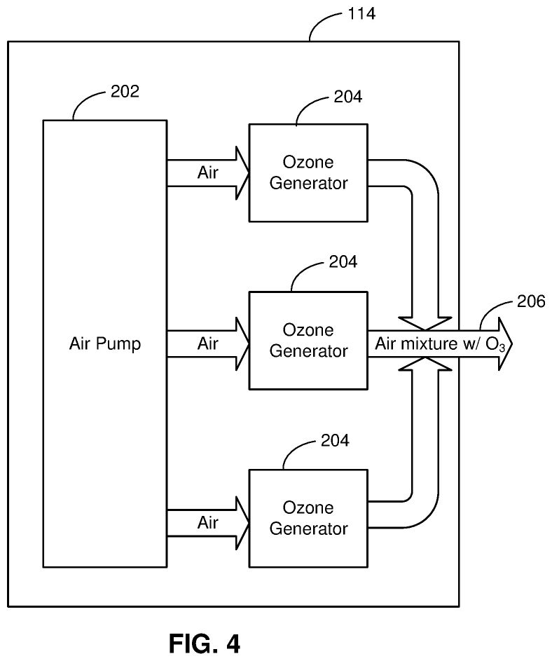

FIG. 4 is a diagram illustrating the ozone supply 114 according to one aspect of the disclosure. The ozone supply 114 includes an air pump 202 and one or more ozone generators 204 for generating ozone (e.g., O₃). In some aspects of the disclosure, the ozone generator 204 may be an ultraviolet light ozone generator, corona discharge ozone generator, or any suitable type of ozone generator. Three exemplary individual ozone generators 204 are shown in FIG. 4. In other aspects of the disclosure, more or fewer ozone generators 204 may be used. In operation, the air pump 202 generates an air stream that is supplied to the ozone generators 204. The air (e.g., atmospheric air) is mixed with ozone generated by the ozone generator 204 to produce an air mixture with ozone 206. The air stream may have a pressure greater than atmospheric pressure or any desired pressure. In one example, the air pressure may be up to 2.5 pounds per square inch (psi) for a small container (e.g., 5 gallon container). In another example, the air pressure may be up to 6 psi for a larger container (e.g., 55-gallon container or larger). The ozone-mixed air stream or ozone may be supplied to the water treatment device 112 (see FIGS. 1-3) to disinfect the water in the container 102. In one aspect of the disclosure, the ozone generator 204 may be connected to the water treatment device 112 by the tubing 116 that carries the ozone-mixed air stream to the lower portion of the container 102. In some aspects of the disclosure, the tubing may be a flexible tube made of any suitable material and length such that the water treatment device 112 can be positioned at the lower portion of the container 102. Because the water treatment device 112 is placed at the bottom or lower portion of the container 102, the ozone (e.g., ozone mixed air) distributed by the water treatment device 112 raises up from the bottom of the container 102 to the water surface so as to increase the cleaning and disinfecting effect of the ozone.

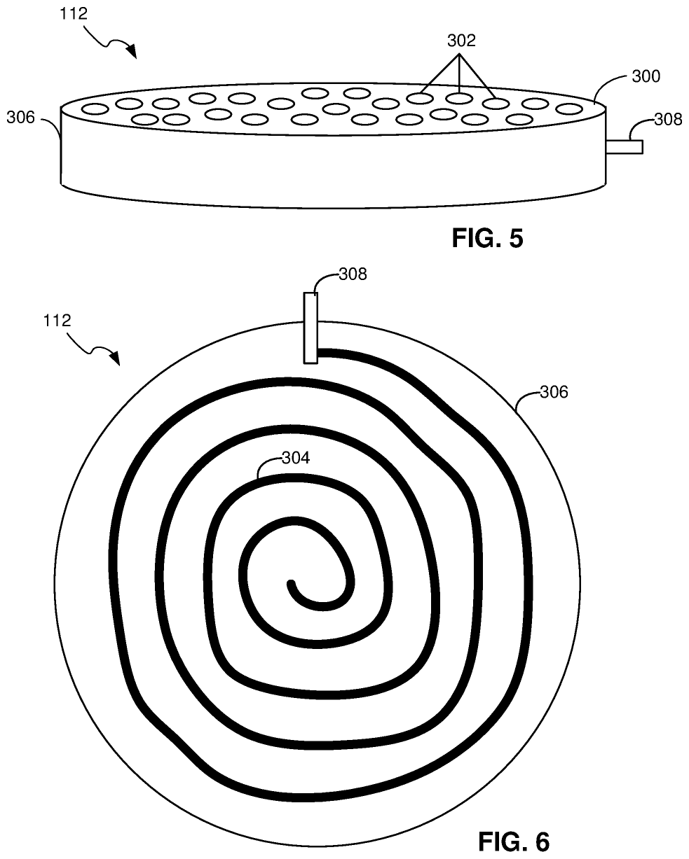

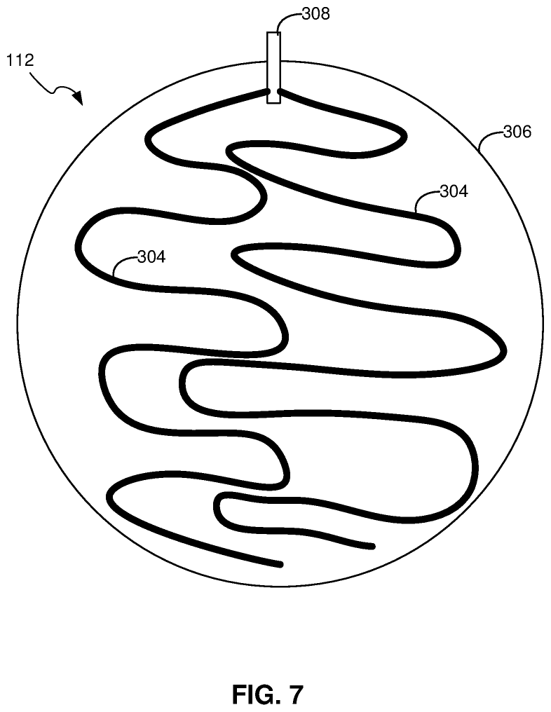

FIG. 5 is a diagram illustrating a perspective view the water treatment device 112 according to one aspect of the disclosure. FIG. 6 is a diagram illustrating a top view of the water treatment device 112 of FIG. 5. The water treatment device 112 has a cover 300 and a housing 306. The cover 300 is not shown in the top view in FIG. 6. In some examples, the cover 300 and housing 306 may be made of stainless steel or any material that is corrosion resistant.

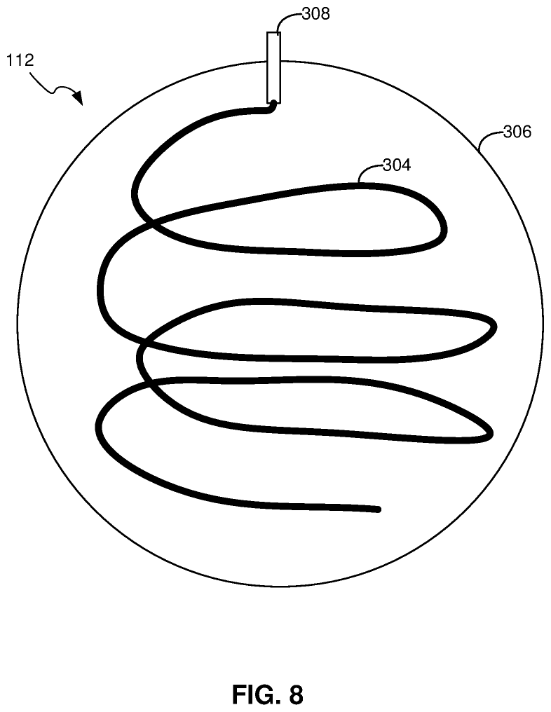

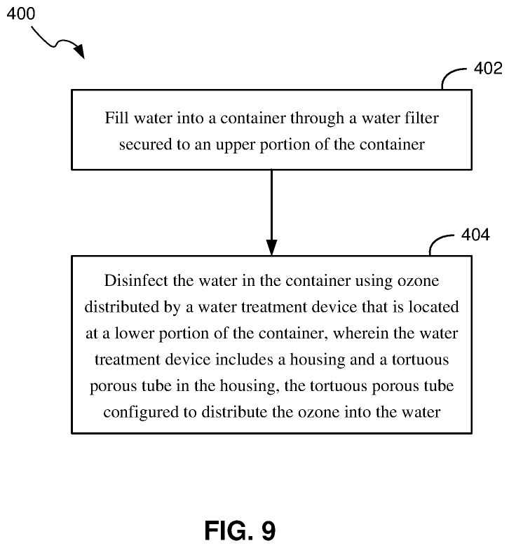

The cover 300 has a plurality of openings 302 that allow ozone or ozone-mixed air to egress or escape from the device during operation. The water treatment device 112 includes a tortuous porous tube 304 in the housing 306. The porous tube 304 may be connected to the hose or pipe 116 (see FIG. 2) via an inlet, connector, or coupler 308. The porous tube 304 when extended or unwound is substantially longer (e.g., 3 times or more) than the length and/or width of the housing 306. In one example, the porous tube 304 when extended has a length (e.g., 65 inches) five times longer than the length and/or width of the housing 306. In this particular example, the porous tube 304 is coiled or wound to fit into the housing 306. In some aspects of the disclosure, the housing 306 may have other openings (not shown) on the side and bottom. The openings allow water to enter the water treatment device 112 when it is submerged in water such that the porous tube 304 is submerged in water during operation. The shape of the porous tube 304 shown in FIG. 6 is illustrative in nature only, and the porous tube 304 may have other tortuous configurations. In some aspects of the disclosure, referring to FIG. 7, the water treatment device 112 may be configured with two or more porous tubes 304. FIG. 8 is a diagram illustrating the water treatment device 112 with a porous tube 304 in a coiled shape according to one aspect of the disclosure.

In some aspects of the disclosure, the porous tube 304 may have other tortuous configurations like a wound shape, helical shape, bend shape, circular shape, rolled shape, spiral shape, and the like. By maximizing the length of the porous tube 304 that can be fitted into the housing 306, a ratio of the surface area of the porous tube 304 to the volume of the housing can be increased. Therefore, the efficiency or rate of ozone diffusion of the water treatment device 112 can be increased. In some aspects of the disclosure, the water treatment device 112 can provide about 100 liters of ozone-mixed air per minute. In one aspect of the disclosure, the water treatment device 112 may be 13 inches in diameter and 1¾ inches in depth. In one example, the water treatment device can provide up to 40 LPM (liter per minute) of ozone-mixed air to a small container (e.g., 5-gallon container). In another example, the water treatment device can provide up to about 110 LPM of ozone-mixed air to a large container (e.g., 55 gallon container). During operation, the porous tube 304 can diffuse a large amount of ozone-mixed air bubbles into the water to achieve effective and efficient disinfection of the water using ozone.

FIG. 8 is a flow chart illustrating a method 400 of disinfecting water using ozone according to one aspect of the disclosure. In one example, the method 400 may be performed using the water treatment system 100 or any suitable water treatment system. At block 402, the method fills water into a container 102 through a water filter 108 secured to an upper portion of the container 102. For example, the water filter 108 may be secured under a cover 110 on the container 102. At block 404, the method disinfects the water in the container using ozone distributed by a water treatment device 112 that is located at a lower portion of the container. The water treatment device 112 (see FIGS. 5 and 6) includes a housing 306 and a tortuous porous tube 304 in the housing. The tortuous porous tube 304 is configured to distribute the ozone into the water in the container.

While the above description contains many specific embodiments of the invention, these should not be construed as limitations on the scope of the invention, but rather as examples of specific embodiments thereof. Accordingly, the scope of the invention should be determined not by the embodiments illustrated, but by the appended claims and their equivalents.

What is claimed is:

1. A water treatment system using ozone, comprising:
a container for holding water;
an ozone supply configured to supply ozone; and
a water treatment device secured at a lower portion of the container and configured to disinfect the water using the ozone in the container, the water treatment device comprising a housing having a plurality of openings and a tortuous porous tube in the housing, the tortuous porous tube configured to receive the ozone from the ozone supply and distribute the ozone into the water.

2. The water treatment system of claim 1, wherein the tortuous porous tube when extended is longer than the water treatment device in at least one of a length direction or a width direction.

3. The water treatment system of claim 2, wherein the tortuous porous tube when extended is at least three times longer than the water treatment device in at least one of a length direction or a width direction.

4. The water treatment system of claim 1, wherein the tortuous porous tube comprises at least two individual porous tubes.

5. The water treatment system of claim 1, wherein the tortuous porous tube is configured in at least one of:
a coiled shape;
a helical shape;
a spiral shape;
a circular shape; or
a wound shape.

6. The water treatment system of claim 1, wherein the ozone supply is located on an upper portion of the container.

7. The water treatment system of claim 6, wherein the ozone supply comprises a plurality of individual ozone generators.

8. The water treatment system of claim 1, further comprising a water filter configured to filter the water entering the container.

9. The water treatment system of claim 8, wherein the ozone supply and the water filter are secured to a top cover on the container.

10. The water treatment system of claim 1, further comprising a fill value inside the container, the fill value configured to automatically control a water flow of the water into the container.

11. The water treatment system of claim 1, further comprising:
a valve connected to the lower portion of the container and configured to control a flow of the water released from the container.

12. A method of treating water using ozone, comprising:
filling water into a container through a water filter secured to an upper portion of the container; and
disinfecting the water in the container using ozone distributed by a water treatment device that is located at a lower portion of the container, wherein the water treatment device comprises a housing and a tortuous porous tube in the housing, the tortuous porous tube configured to distribute the ozone into the water.

13. The method of claim 12, wherein the disinfecting the water comprises supplying ozone to the tortuous porous tube that when extended is longer than the water treatment device in at least one of a length direction or a width direction.

14. The method of claim 13, wherein the tortuous porous tube when extended is at least three times longer than the water treatment device in at least one of a length direction or a width direction.

15. The method of claim 13, wherein the disinfecting the water comprises supplying ozone to at least two individual porous tubes of the tortuous porous tube.

16. The method of claim 12, wherein the disinfecting the water comprises supplying the ozone to the tortuous porous tube that is configured in at least one of:
a coiled shape;
a helical shape;
a spiral shape;
a circular shape; or
a wound shape.

17. The method of claim 12, further comprising generating the ozone using an ozone supply located on an upper portion of the container.

18. The method of claim 17, wherein the generating the ozone comprises generating the ozone using a plurality of individual ozone generators.
